(19) United States
(12) Patent Application Publication
(10) Pub. No.: US 2023/0098749 A1
(43) Pub. Date: Mar. 30, 2023

(54) Plasma Sources for Generating Cold Plasma

(71) Applicant: The Regents of the University of California, Oakland, CA (US)

(72) Inventors: Richard Edward Wirz, Los Angeles, CA (US); Zhitong Chen, Los Angeles, CA (US)

(73) Assignee: The Regents of the University of California, Oakland, CA (US)

(21) Appl. No.: 17/904,468

(22) PCT Filed: Mar. 2, 2021
(86) PCT No.: PCT/US21/20463

§ 371 Date: Aug. 17, 2022

Related U.S. Application Data
(60) Provisional application No. 62/984,267, filed on Mar. 2, 2020.

(51) Int. Cl. A61K 41/00; A61K 33/00; A61K 9/08; B01J 19/08

(52) U.S. Cl. CPC A61K 41/0004; A61K 33/00; A61K 9/08; B01J 19/088; B01J 2219/0801; B01J 2219/0896; B01J 2219/0875; B01J 2219/0824

(57) ABSTRACT

Apparatus and systems describe a plasma source to generate cold plasma solution for use in hospital, home, office, and other locations. The plasma source employs air, gas, and/or vapor discharging in a high voltage electric field, which interact to obtain plasma solution for oral health. The plasma source may include power supply, plasma generator, liquid container, liquid aerosolizer/vaporizer or liquid disturber, reservoir, collector, and intelligent control center.

### CROSS-REFERENCE TO RELATED APPLICATIONS

The current application claims the benefit of and priority to U.S. Provisional Patent Application No. 62/984,267 entitled “Plasma Sources for Oral Health” filed Mar. 2, 2020. The disclosure of U.S. Provisional Patent Application No. 62/984,267 is hereby incorporated by reference in its entirety for all purposes.

### FIELD OF THE INVENTION

The present invention generally relates to plasma source apparatus and systems for generating cold plasma; and more particularly to utilizing cold plasma sources for oral health applications and procedures.

### BACKGROUND OF THE INVENTION

Plasma is the fourth state of matter, contrasted with other states: solid, liquid, and gas. It is an ionized gas containing charged and neutral species, radicals, and photons, which comprises over 99% of our visible universe. Cold plasmas have received considerable attention due to their great promise in biomedicine. Cold plasma is effective, economical, and environmentally safe, and it can initiate, promote, control, and catalyze various complex behaviors and responses in biological systems due to its unique properties. The efficacy of cold plasma is derived from its components, especially reactive oxygen species (ROS) and reactive nitrogen species (RNS). Active oxygen species can promote a “plasma killing effect”, while nitric oxide can produce “plasma healing”.

### BRIEF SUMMARY OF THE INVENTION

Apparatuses and systems for plasma sources for generating cold plasma are illustrated.

One embodiment of the invention includes a plasma source comprising at least a power system; at least a plasma generator; at least a liquid container; at least a liquid agitator, wherein the liquid agitator uses liquid agitation and transports a convection; at least a control center; and the plasma source generates a cold plasma solution.

In a further embodiment, the cold plasma solution comprises at least one reactive oxygen species and at least one reactive nitrogen species.

In another embodiment, the power system comprises a power supply.

In a still further embodiment, the power supply is an AC power supply or a DC power supply.

In another further embodiment, the power supply is a battery or a plugin.

In an additional embodiment, the battery is a 12 V battery.

In a yet further embodiment, the power system comprises a power processor.

In yet another embodiment, the power processer converts a power input into a current, a voltage, a thermal source, or a mechanical source.

In a further embodiment again, the plasma generator comprises at least two electrodes.

In another embodiment again, the plasma generator comprises at least one stage of electrodes to facilitate at least one ionization stage as a gas passes through a discharge region.

In a still yet another embodiment, the shape of the electrode is cylindrical, conical, square, annular, or rectangular.

In still yet another embodiment, the plasma generator comprises at least one gas source.

In a still further embodiment again, the gas source is a pressurized vessel.

In a still further embodiment, the gas source supplies a gas to the plasma generator and the gas is air, hydrogen, helium, neon, argon, krypton, xenon, radon, iodine, chlorine, oxygen, nitrogen, and any combinations thereof.

In yet another further embodiment, the plasma generator generates a plasma from the supplied gas.

In still another further embodiment, the gas is flavored with at least one flavoring.

In another further embodiment, the gas is supplied to the plasma generator via pressurization, forced convection, entrainment, and any combinations thereof.

In still yet another embodiment, the liquid container comprises a liquid source.

In another embodiment, the liquid source comprises at least one of water, deionized water, sparkling water, water with at least one dissolved gas, and liquid with at least one flavoring.

In yet another embodiment, the liquid agitator is a liquid aerosolizer, a liquid vaporizer, or a liquid disturber.

In a further embodiment, the liquid agitator generates a liquid vapor.

In an additional further embodiment, the convection is a sonic convection, a vibrational convection, a thermal convection, a mechanical convection, or any combinations thereof.

In yet another embodiment, the control center has an interface.

In another further additional embodiment, the interface is on a device selected from the group consisting of computer, tablet, and phone.

In still yet another further embodiment, the plasma source further comprises a receptacle cup.

In still another further embodiment again, the plasma source further comprises a transfer tube.

Another further embodiment includes a method of generating a cold plasma solution comprising:
turning on a power supply;
adding a liquid to a liquid container;
agitating the liquid via a liquid aerosolizer, a liquid vaporizer, or a liquid disturber;
transferring the agitated liquid through a transfer tube to a mixing chamber;
feeding and ionizing a gas feed to form a plasma;
transferring the plasma to the mixing chamber;
mixing the agitated liquid and the plasma;
condensing the mixed agitated liquid and the plasma into a cold plasma solution; and
dispensing the cold plasma solution into a receptacle cup.

In yet another further embodiment, the cold plasma solution comprises at least one reactive oxygen species and at least one reactive nitrogen species.

In a still further embodiment, the power supply is an AC power supply or a DC power supply.

In another embodiment again, the power supply is a battery or a plugin.

In a further additional embodiment, the battery is a 12 V battery.

In another additional embodiment, the power supply comprises a power processor.

In a still yet further embodiment, the power processer converts a power input into a current, a voltage, a thermal source, or a mechanical source.

In still yet another embodiment, the gas feed is from a pressurized vessel.

In a still further embodiment again, the gas feed is air, hydrogen, helium, neon, argon, krypton, xenon, radon, iodine, chlorine, oxygen, nitrogen, and any combinations thereof.

In still yet further embodiment, the gas feed is flavored with at least one flavoring.

In a still further embodiment again, the gas feed is fed to generate the plasma via pressurization, forced convection, entrainment, and any combinations thereof.

In still another embodiment again, the liquid is at least one of water, deionized water, sparkling water, water with at least one dissolved gas, and liquid with at least one flavoring.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a Type 1 plasma source in accordance with embodiments.

FIG. 2 illustrates a Type 2 plasma source in accordance with embodiments.

FIG. 3 illustrates a representative plasma discharge in air at atmospheric pressure in accordance with embodiments.

FIG. 4 illustrates a representative emission spectrum for plasma-generated reactive oxygen species and reactive nitrogen species in accordance with embodiments.

FIGS. 5A-5C illustrate various methods to generate plasma.

### DETAILED DESCRIPTION OF THE INVENTION

Turning now to the drawings, plasma source apparatus and systems for generating cold plasma and procedures are described. In many embodiments, the plasma sources generate cold plasma and/or cold plasma solution. The plasma sources in accordance with several embodiments employ air, gas and/or vapor discharging in a high voltage electric field, which interacts with a liquid to obtain a plasma solution. In several embodiments, cold plasma and/or cold plasma solution refer to a plasma and/or a plasma solution where the temperature of the individual constituents are different from each other. Cold plasma in accordance with certain embodiments is a source of high-temperature electrons at ambient conditions including room temperature and atmospheric pressure. In some embodiments, cold plasma produces many reactive species including (but not limited to) reactive oxygen species (ROS) and reactive nitrogen species (RNS). Many embodiments provide cold plasma sources for use in locations including (but not limited to): hospitals, clinics, laboratories, homes, and offices.

In many embodiments, the plasma source for generating cold plasma comprises: at least one power system, at least one plasma generator, at least one liquid container, at least one liquid agitator, and at least an intelligent control center. A number of embodiments include at least a reservoir and/or at least a collector in the plasma source. In some embodiments, the power system can be an AC or a DC electrical power supply including (but not limited to): battery and plugin. Some embodiments use 12 V batteries. In several embodiments, the power system can be a power processor that converts power input into an appropriate source including (but not limited to): current, voltage, thermal source, and mechanical source, to enable the plasma source operation. The power system of the plasma source in accordance with several embodiments can be adapted to be used everywhere around the world by changing the wall plug. In many embodiments, the plasma generator includes at least two electrodes. In several embodiments, the plasma generator includes multiple stages and/or pairs of electrodes to facilitate multiple ionization stages as the gas or vapor passes through the discharge region. In a number of embodiments, the shape of electrodes for the plasma source include (but are not limited to): cylindrical, conical, square, rectangular, and annular. The liquid container in accordance with several embodiments contains a liquid source including (but not limited to): water, deionized water, water with at least one dissolved gas, and liquid with flavorings. Some embodiments provide that the liquid agitator can be a liquid aerosolizer, a liquid vaporizer, or a liquid disturber. In several embodiments, the liquid agitator uses liquid agitation and transport convection including (but not limited to): sonic convection, vibrational convection, thermal convection, and mechanical convection. Many embodiments implement ambient air and/or gas feed in the plasma sources. Several embodiments implement a gas source that uses at least one of pressurization, forced convection, and entrainment to move gas through the plasma source. A number of embodiments provide that there is a means for forced convection of at least one of ambient air and gas through the plasma source. Several embodiments implement the combination of a vapor and/or a liquid feed system to generate a cold plasma solution. Many embodiments provide that the plasma sources are approximately the size of a small home coffee machine.

In many embodiments, the plasma source systems can be operated in-person, remotely, and/or on a schedule. Several embodiments provide that the plasma sources can be interfaced directly and/or via smart device interfaces including (but not limited to) computers, tablets, and phones. The plasma source interfaces in accordance with some embodiments may include and/or add additives to improve the flavor of the plasma-activated solution before, during, or after activation.

Cold plasma, with room temperature and normal atmospheric pressure, has a wide range of biomedical applications. Cold plasma is able to efficiently kill bacteria, yeast, mold, and other hazardous microorganisms that may be difficult to inactivate by traditional methods. Cold plasma can be employed for decontamination and sterilization of water, air, food, and living tissues without causing damage or other side effects. Additionally, cold plasma can stop bleeding, treat intestinal ulcers, remove injured area, accelerate wound healing, and others. For biomedical applications, cold plasma has been applied to wound healing, sterilization, blood coagulation, skin disease, cancer therapy, immunotherapy, and so on.

In many embodiments, the plasma source can be implemented in oral health applications, medical and biomedical applications, and agriculture applications. Many embodiments provide a safe, novel, small-volume plasma source to enable a wide range of applications for oral health. Several embodiments provide the plasma source for dental health. The integration of cold plasma technology into the plasma source in accordance with some embodiments enable the prevention and treatment of dental diseases. Some embodiments incorporate an intelligent plasma source that possess functions of at least one of mouthwash, gum, and teeth whitening technologies. Several embodiments provide that the plasma source can be used to improve breath. The easy and convenient cold plasma source, in accordance with a number of embodiments, can be adapted for use in places including (but not limited to): hospitals, clinics, labs, homes, and offices. Some embodiments incorporate a simplified plasma generation system using ambient air for at-home use in the plasma source system. Such embodiments provide a faster and simpler one-step process to replace other at-home dental care devices and improve existing technology and processes.

Many embodiments combine vapor and/or liquid feed systems to generate a plasma solutions for home use to improve oral health. Some embodiments use ambient air in the local environment as feed gas for the plasma source. Several embodiments use designated sources including (but not limited to) pressurized vessels as the feed gas. Certain embodiments use designated feed gas including (but not limited to): air, hydrogen, helium, neon, argon, krypton, xenon, radon, iodine, chlorine, oxygen, nitrogen, and any combinations thereof. Some embodiments use canned or central pressurized air as the feed gas. A number of embodiments implement liquid solutions that enable dental treatments including (but not limited to): mouthwash, and demonstrate additional efficacy when activated by the plasma. Many embodiments use water or liquid sources from at least one of: tap, bottle, store bought, sparkling water, or any purification system. The plasma solution generated by the plasma source in accordance with several embodiments can be flavored via the air and/or the liquid for improved taste.

Many embodiments implement plasma sources in medical and biomedical applications. The cold plasma solution obtained from the plasma source in accordance with some embodiments can be applied in secondary medical uses including (but not limited to): treatment of wound healing, skin diseases, and sterilization.

Some embodiments implement plasma sources in agricultural applications. In several embodiments, the cold plasma solution obtained from the plasma source can be applied to crops as fertilizer, to seeds directly to increase germination and yield, and to foods to increase their safety and quality. Applying plasma solution to foods can increase their safety and quality through nonthermal pasteurization or sterilization. Certain embodiments provide that application of cold plasma solution can reduce food waste and efficiently degrade pesticides and mycotoxins as well as inactivate pests.

### Plasma Sources

Plasma's unique physical and chemical properties enable medical applications that include treatments of wound healing, dental cavities, sterilization, regenerative medicine, and so on. (See e.g., Plasma Process. Polym., 2016, 13, 1151-1156, and Free Radic. Biol. Med., 2019, 130, 71-81; the disclosures of which are incorporated herein by reference). Plasma interaction with tissue depends on plasma-generated chemical elements such as ROS and RNS. (See e.g., Biointerphases, 2016, 11, 031010, and J. Phys. D: Appl. Phys., 2017, 50, 015208; the disclosures of which are incorporated herein by reference). Plasma-generated reactive species and UV photons have a sterilization effect. (See e.g., Pure Appl. Chem., 2002, 74, 349-358, the disclosure of which is incorporated herein by reference). The unique ability of plasma to discharge with different modes and to form coherent structures allows instantaneous modulation of electric field, reactive species, and charged particles. (See e.g., Sci. Rep., 2017, 7, 12163, the disclosure of which is incorporated herein by reference). A large-area plasma decontamination device has been used for decontamination of biological and chemical incidents in medical, industrial and civilian situations. (See e.g., U.S. Pat. No. 8,377,388 B2, the disclosure of which is incorporated herein by reference). Plasma jets and/or dielectric barrier discharge plasma are also employed for cold plasma medical treatments. (See e.g., patent application No. 2014/018071A1 to Riley, and U.S. patent application No. 2014/0378892A1 to Keidar et al.; the disclosure of which are incorporated herein by reference). A device has been developed for use in treating internal organs with plasma or plasma-induced species (See e.g., U.S. patent application No. 2013/0053762 A1 to Rontal et al., the disclosure of which is incorporated herein by reference).

The diversity of the mechanisms of action of cold plasma, and the flexibility as a standalone technology or one that can integrate with other technologies, provide a rich resource for driving innovative solutions. Cold plasma is increasingly investigated for translation to a plethora of issues in the sterilization and dental sectors. An improved method for cleaning using plasma has been proposed for medicine, dentistry, and relates to cleaning and decontamination of instruments. It is also used in food preparation, in which contaminated articles are exposed to solvent and then exposed to the plasma for sterilization and cleaning. (See e.g., JP patent application No. 2008/535527A, the disclosure of which is herein incorporated by reference). On the other hand, plasma disinfection and purification devices have been developed to provide dental care, where the plasma can sterilize the air around the bed to effectively kill bacteria, viruses and other harmful substances. (See e.g., CN patent application No. 204319350U, the disclosure of which is herein incorporated by reference). Another dental plasma apparatus developed for the oral cavity, collects the oral state information from the bio-signal collection unit and analyzes data to make a decision on generating right plasma intensity for oral state. (See e.g., KR patent application No. 2019/0102875A, the disclosure of which is herein incorporated by reference). A process for dry sterilization of medical or dental device and materials, where these materials are subjected to an electrical discharge in a gaseous atmosphere to produce an active low temperature plasma for surface sterilization and treatment of the devices and materials has also been developed. (See e.g., U.S. Pat. No. 5,087,418, the disclosure of which is herein incorporated by reference).

Many embodiments implement a plasma source that represents a significant advance over conventional sources. While the conventional plasma techniques may be large apparatus that require separate maintenance, training, and coordination, the plasma source described in embodiments is of a small volume and is safe, making it simple, convenient, and comfortable to use. In some embodiments, the plasma sources show high standardization, reparability, reliability, and repeatability.

### Plasma Sources for Generating Cold Plasma

For a more complete understanding of the present invention and the advantage, reference is now made to the following description and the accompanying drawings. Two specific implementations of the plasma sources are presented below. The following diagrams are included to demonstrate the mechanisms and advantages of the proposed plasma sources. While specific terms and implementations are mentioned to provide context and detail, it should not be limited to the specifics mentioned here. This design should be considered as encompassing all technical equivalents operating in a similar fashion with similar purpose.

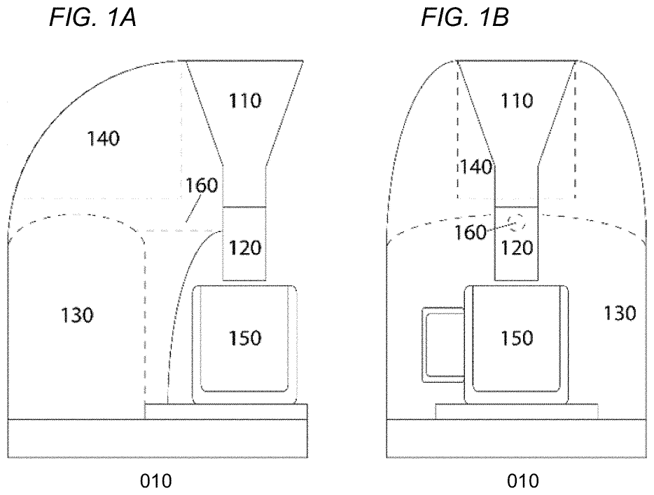

Many embodiments implement plasma sources with variable features. A profile side view and front view of a Type 1 plasma source in accordance with an embodiment of the invention is illustrated in FIG. 1A and FIG. 1B. The Type 1 plasma source (010), in accordance with some embodiments, identifies six primary features: the plasma generator and gas feed system (110); the vapor/plasma mixing chamber and condenser mechanism (120); the liquid reservoir with integrated aerosolizer/vaporizer (130), the power, control, and networking electronics (140); the receptacle cup for the plasma-infused liquid (150), and the liquid vapor transfer tube (160).

In many embodiments, water and/or other appropriate liquid can be initially added to the reservoir (130). When activated via a switch, control, or network command via the electronics (140), liquid within the reservoir can be aerosolized (such as via vibrational excitation) and travels through the transfer tube (160) to the mixing chamber (120). At the same time, ambient air, feed gases, or a mixture of the two can be drawn in through the top of the plasma generator and gas feed system (110) and partially ionized into plasma within the generator. Some embodiments implement the flow rate of the inlet gas should sustain the plasma generation. The gas-fed plasma then enters the mixing chamber (120) where it mixes with the water vapor. The condensing mechanism within (120) condenses the now-plasma-infused liquid into primarily liquid form. The liquid can then be fed along with the remaining gases downwards into the waiting receptacle cup (150). Then, plasma solution containing ROS, RNS, and other plasma-activated species can be obtained. In many embodiments, the plasma source and gas source may be detachable. In several embodiments, the attachment may be extendable to accommodate larger reservoir or a conveyor of reservoirs.

Some embodiments provide a process of using cold plasma solution generated by the Type 1 plasma source. The process in accordance with certain embodiments include: pour 20 milliliters of plasma solution into the mouth; swish for about at least 30 seconds; gargle in the mouth during swishing; spit the solution out in the sink. The process can be repeated two or three times. The plasma-activated species can stay active for at least 3 minutes in accordance with some embodiments. In several embodiments, the cold plasma solution can be stored in a non-reactive vessel for later uses.

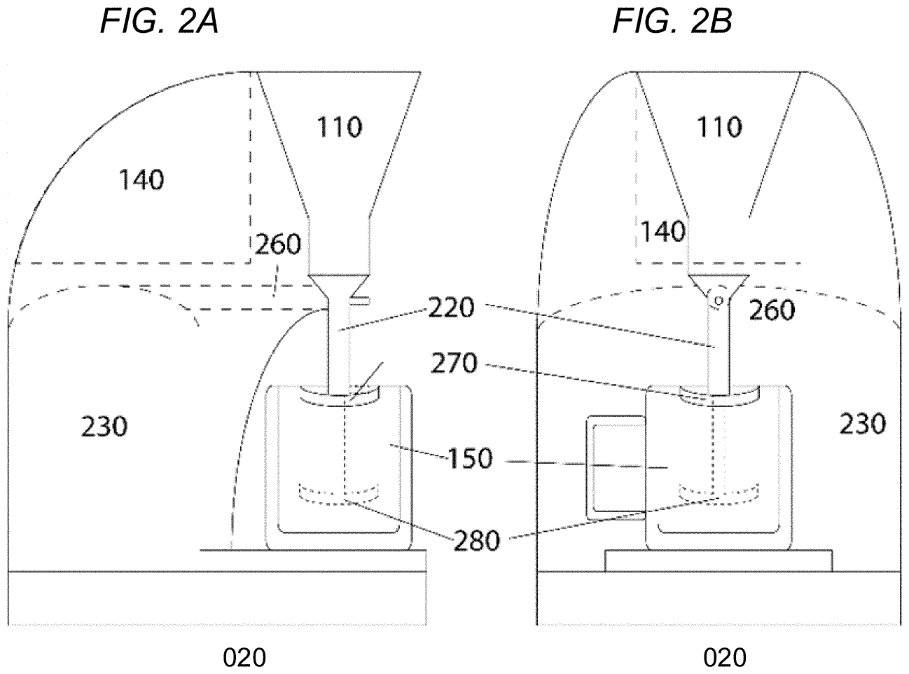

A profile side view and front view of a Type 2 plasma source in accordance with an embodiment of the invention is illustrated in FIG. 2A and FIG. 2B. The Type 2 plasma source (020), in accordance with some embodiments, identifies eight primary features: the plasma generator and gas feed system (110); the gas and liquid delivery mechanism (220); the liquid reservoir with pump (230), the power, control, and networking electronics (140); the receptacle cup for the plasma-infused liquid (150); the liquid transfer tube (260); the gas/liquid spout and mixer in a raised configuration (270); and the gas/liquid spout and mixer in a lowered configuration (280).

In many embodiments, water and/or other appropriate liquid can be initially added to the reservoir (230). The gas/liquid spout (270) may initially be in the raised position. A receptacle (150) can be placed beneath the spout (270), and the spout can be lowered into the receptacle (280). When activated via the lowering of the spout, switch, control, or network command via the electronics (140), liquid within the reservoir can be drawn in via pump and travels through the transfer tube (260) to the delivery mechanism (220). At the same time, ambient air and/or gas is drawn in or fed through the top of the plasma generator and feed (110) and ionized into plasma within the generator. Some embodiments implement a flow rate of the inlet gas that sustains the plasma generation. The gas-fed plasma can then enter the delivery mechanism (220). Within the delivery mechanism, liquid and gas-fed plasma can be kept separate and channeled down to the lowered gas/liquid spout and mixer (280). Upon the end of the spout (280), liquid is delivered directly into the receptacle, while the gas-fed plasma is channeled into the mixer portion, which diffuses the gas-fed plasma into the receptacle (150) through small holes. The spout (280) can then be raised (270). The receptacle (150) containing the plasma-infused liquid can be removed. Then, plasma solution containing ROS and RNS can be obtained. In many embodiments, the plasma source and gas source may be detachable. In several embodiments, the attachment may be extendable to accommodate larger reservoir or a conveyor of reservoirs.

Some embodiments provide a process of using cold plasma solution generated by the Type 2 plasma source. The process in accordance with certain embodiments include: pour 20 milliliters of plasma solution into the mouth; swish for about at least 30 seconds; gargle in the mouth during swishing; spit the solution out in the sink. The process can be repeated two or three times. The plasma-activated species can stay active for at least 3 minutes in accordance with some embodiments. In several embodiments, the plasma solution can be stored in a non-reactive vessel for later uses.

Many embodiments implement development of the plasma sources with more functions and intelligent systems. Some embodiments provide options for various customer requirements or for large scale manufacturing of plasma activated media. Several embodiments provide mechanisms of plasma medicine and plasma agriculture that are based on accurately analyzing lab experimental and clinical data or personal, commercial, or industrial use. Many embodiments implement the plasma sources to apply to more fields including (but not limited to): space missions and remote deployments.

Many embodiments include the plasma sources are approximately the size of a small home coffee machine. Plasma solution obtained from both type plasma sources also can be applied for wound healing, skin diseases, sterilization, and other medical applications. In addition, plasma solution can be applied to crops as fertilizer and to seeds directly to increase germination and yield. Applying plasma solution to foods will increase their safety and quality through nonthermal pasteurization or sterilization. It also can reduce food waste and efficiently degrade pesticides and mycotoxins as well as inactivate pests. Professional and experts can recognize that numerous uses of the plasma sources are possible. The objective is the production of plasma sources, which are not only for preventing and treating oral diseases and improving breath but also for teeth whitening, foods, sterilization, agricultural applications, medical applications, and other biomedical applications.

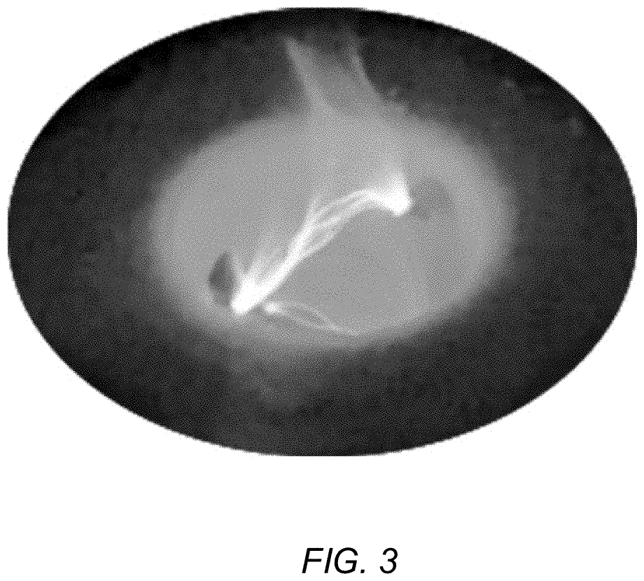

Plasma discharge in air at atmospheric pressure in accordance with an embodiment of the invention is illustrated in FIG. 3.

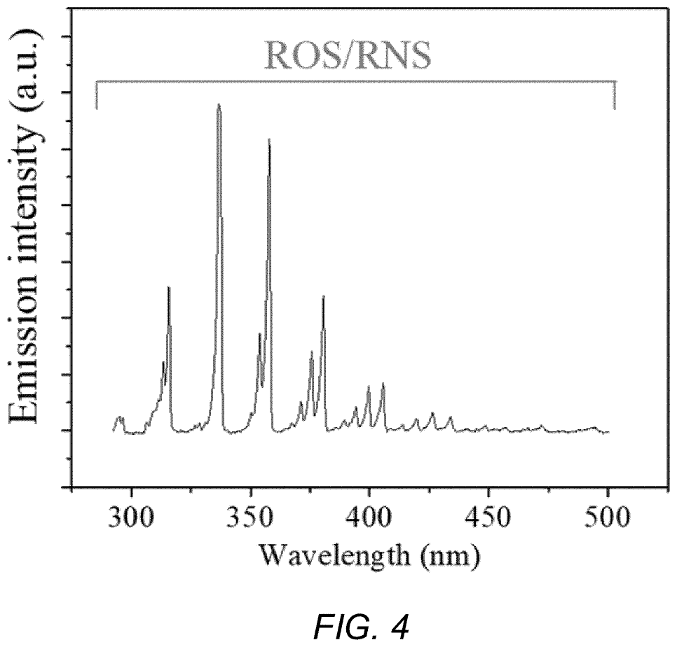

Spectral signature of reactive oxygen species (ROS) and reactive nitrogen species (RNS) generated by plasma sources in accordance with an embodiment of the invention is illustrated in FIG. 4.

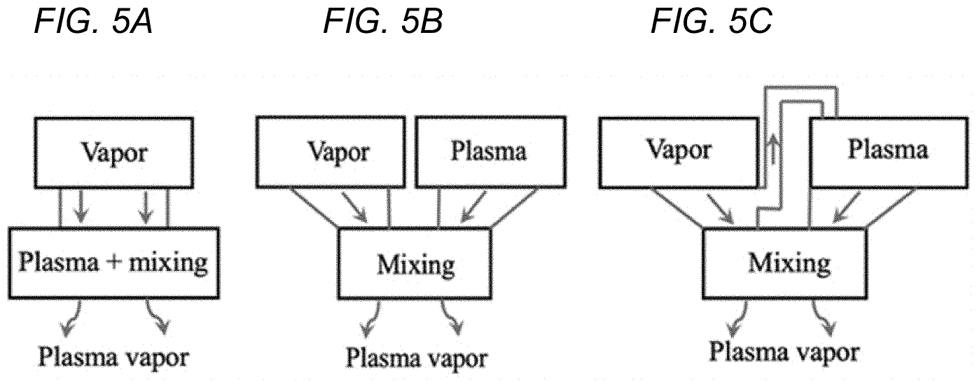

Many embodiments provide various methods to produce cold plasma using plasma sources. The various approaches to produce cold plasma vapor provide a variety of options for plasma vapor composition and ensure a long life for the plasma generation stage. In one method, vapor can directly enter the plasma generator and then spray out as plasma vapor. A method of generating plasma from vapor is illustrated in FIG. 5A. The vapor infused air and/or gas enters the plasma generator that contains the plasma generating electrodes and is then sprayed out or ejected directly as plasma vapor. The mixing chamber may be omitted entirely as mixing may take place during the plasma generation process.

In another method, gaseous plasma and vapor can enter the mixing chamber and generate plasma vapor. A method of generating plasma with plasma and vapor is illustrated in FIG. 5B. Gaseous plasma including (but not limited to) in the air and other gas can be formed and combined with vapor from the liquid including (but not limited to) water entering a mixing chamber to provide plasma vapor.

In another method, a fraction of the vapor can be injected into the plasma generator, while the rest of the vapor can mix with the plasma vapor in a mixing chamber to produce a plasma vapor of the desired concentration. A method of generating plasma with plasma and mixture of plasma and vapor is illustrated in FIG. 5C. FIG. 5C illustrates a combination of the delivery schemes where a fraction of the liquid vapor is injected into the plasma generator, while the rest of the liquid vapor mixes with the plasma vapor in a mixing chamber to produce a plasma vapor of the desired concentration.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A plasma source comprising:
at least a power system;
at least a plasma generator;
at least a liquid container;
at least a liquid agitator, wherein the liquid agitator uses liquid agitation and transports a convection;
at least a control center; and
wherein the plasma source generates a cold plasma solution.

2. The plasma source of claim 1, wherein the cold plasma solution comprises at least one reactive oxygen species and at least one reactive nitrogen species.

3. The plasma source of claim 1, wherein the power system comprises a power supply.

4. The plasma source of claim 3, wherein the power supply is an AC power supply or a DC power supply.

5. The plasma source of claim 4, wherein the power supply is a battery or a plugin.

6. The plasma source of claim 5, wherein the battery is a 12 V battery.

7. The plasma source of claim 1, wherein the power system comprises a power processor.

8. The plasma source of claim 7, wherein the power processer converts a power input into a current, a voltage, a thermal source, or a mechanical source.

9. The plasma source of claim 1, wherein the plasma generator comprises at least two electrodes.

10. The plasma source of claim 1, wherein the plasma generator comprises at least one stage of electrodes to facilitate at least one ionization stage as a gas passes through a discharge region.

11. The plasma source of claim 9, wherein the shape of the electrode is cylindrical, conical, square, annular, or rectangular.

12. The plasma source of claim 1, wherein the plasma generator comprises at least one gas source.

13. The plasma source of claim 12, wherein the gas source is a pressurized vessel.

14. The plasma source of claim 12, wherein the gas source supplies a gas to the plasma generator and the gas is air, hydrogen, helium, neon, argon, krypton, xenon, radon, iodine, chlorine, oxygen, nitrogen, and any combinations thereof.

15. The plasma source of claim 14, wherein the plasma generator generates a plasma from the supplied gas.

16. The plasma source of claim 14, wherein the gas is flavored with at least one flavoring.

17. The plasma source of claim 14, wherein the gas is supplied to the plasma generator via pressurization, forced convection, entrainment, and any combinations thereof.

18. The plasma source of claim 1, wherein the liquid container comprises a liquid source.

19. The plasma source of claim 12, wherein the liquid source comprises at least one of water, deionized water, sparkling water, water with at least one dissolved gas, and liquid with at least one flavoring.

20. The plasma source of claim 1, wherein the liquid agitator is a liquid aerosolizer, a liquid vaporizer, or a liquid disturber.

21. The plasma source of claim 20, wherein the liquid agitator generates a liquid vapor.

22. The plasma source of claim 1, wherein the convection is a sonic convection, a vibrational convection, a thermal convection, a mechanical convection, or any combinations thereof.

23. The plasma source of claim 1, wherein the control center has an interface.

24. The plasma source of claim 23, wherein the interface is on a device selected from the group consisting of computer, tablet, and phone.

25. The plasma source of claim 1, further comprising a receptacle cup.

26. The plasma source of claim 1, further comprising a transfer tube.

27. A method of generating a cold plasma solution, comprising:
turning on a power supply;
adding a liquid to a liquid container;
agitating the liquid via a liquid aerosolizer, a liquid vaporizer, or a liquid disturber;
transferring the agitated liquid through a transfer tube to a mixing chamber;
feeding and ionizing a gas feed to form a plasma;
transferring the plasma to the mixing chamber;
mixing the agitated liquid and the plasma;
condensing the mixed agitated liquid and the plasma into a cold plasma solution; and
dispensing the cold plasma solution into a receptacle cup.

28. The method of claim 27, wherein the cold plasma solution comprises at least one reactive oxygen species and at least one reactive nitrogen species.

29. The method of claim 27, wherein the power supply is an AC power supply or a DC power supply.

30. The method of claim 27, wherein the power supply is a battery or a plugin.

31. The method of claim 30, wherein the battery is a 12 V battery.

32. The method of claim 27, wherein the power supply comprises a power processor.

33. The method of claim 32, wherein the power processer converts a power input into a current, a voltage, a thermal source, or a mechanical source.

34. The method of 27, wherein the gas feed is from a pressurized vessel.

35. The method of 27, wherein the gas feed is air, hydrogen, helium, neon, argon, krypton, xenon, radon, iodine, chlorine, oxygen, nitrogen, and any combinations thereof.

36. The method of 27, wherein the gas feed is flavored with at least one flavoring.

37. The method of 27, wherein the gas feed is fed to generate the plasma via pressurization, forced convection, entrainment, and any combinations thereof.

38. The method of 27, wherein the liquid is at least one of water, deionized water, sparkling water, water with at least one dissolved gas, and liquid with at least one flavoring.
